(19) United States
(12) Patent Application Publication
(10) Pub. No.: US 2026/0257981 A1
(43) Pub. Date: Sep. 3, 2026

(54) METHOD FOR ACHIEVING SYNERGISTIC EFFECT FOR IMPROVING THERMODYNAMIC STABILITY OF GAS HYDRATES BY USING ORGANIC MIXTURE AS HYBRID PROMOTER

(71) Applicant: SEOUL NATIONAL UNIVERSITY R&DB FOUNDATION, Seoul (KR)

(72) Inventor: Jiwoong SEOL, Seoul (KR)

(21) Appl. No.: 19/653,180

(22) Filed: Apr. 21, 2026

Related U.S. Application Data
(63) Continuation of application No. PCT/KR2024/015728, filed on Oct. 17, 2024.

(30) Foreign Application Priority Data
Oct. 24, 2023 (KR) 10-2023-0142939
Feb. 28, 2024 (KR) 10-2024-0028510

(51) Int. Cl. C07C 49/413

(52) U.S. Cl. CPC C07C 49/413

(57) ABSTRACT

The present disclosure relates to a method for achieving synergistic effect for improving thermodynamic stability of gas hydrates by using organic mixture as hybrid promoter. The hybrid promoter according to embodiments of the present disclosure can significantly reduce the energy required for the overall processes of CH₄ hydrate formation, storage, and transportation, thereby enabling an energy-efficient process.

### CROSS-REFERENCE TO RELATED APPLICATION

This application is a continuation of PCT Application No. PCT/KR2024/015728, filed on Oct. 17, 2024, which claims priorities to Korean Patent Applications Number 10-2023-0142939, filed on Oct. 24, 2023, and 10-2024-0028510, filed on Feb. 28, 2024, all of which are hereby incorporated by reference in their entirety.

### TECHNICAL FIELD

The present disclosure provides a method for achieving synergistic effect for improving thermodynamic stability of gas hydrates by using organic mixture as hybrid promoter.

### BACKGROUND

As the global population continues to expand, worldwide energy demand has been steadily increasing, and the demand for natural gas has also risen consistently over the last several decades. Recently, various factors such as COVID-19 pandemic, war-induced price fluctuations, increased investment in clean and renewable energy, and technological advancements have emerged. Nevertheless, natural gas remains an important energy source. It accounted for approximately 25% of primary energy consumption until the early 2020s, and this proportion is expected to remain stable over the coming decades.

In light of these factors, there is a growing need for sustainable, cost-effective, and safe methods for transporting natural gas. One notable approach involves storing natural gas or pure CH₄ in gas hydrates, which is a method recognized as a potential form of solid natural gas or hydrate-based energy storage (HBES). Also, this concept has been studied for the past 30 years, as disclosed in the prior literature: J. S. Gudmundsson et al., “Storing natural gas as frozen hydrate,” SPE Prod. Fac. 9 (1994) 69-73.

Gas hydrates are substances in which water molecules form cage-like structures through hydrogen bonding and gas molecules are trapped via van der Waals interactions. Pure CH₄ hydrate can store approximately 170 times its volume of CH₄ gas. Further, since water is the primary component, the hydrate is non-explosive and environmentally friendly, making it inherently safe. However, its storage capacity is relatively low compared to liquefied natural gas (LNG) and compressed natural gas (CNG). Therefore, minimizing energy consumption throughout the CH₄ hydrate formation, storage, and transportation processes is essential to achieve competitiveness with LNG and CNG in terms of energy density.

The thermodynamic stability of CH₄ hydrate affects the entire process, including production, storage, and transportation. By applying the self-preservation effect to pure CH₄ hydrate, long-term storage is possible at relatively high temperatures and low pressures. By storing pure methane without additional components, this method achieves superior CH₄ storage capacity compared to systems employing thermodynamic promoters, in which the thermodynamic promoters occupy a portion of the hydrate cages. However, the exact mechanism of the self-preservation effect has not been fully elucidated, and issues concerning operational control and the potential for CH₄ loss have been raised. Accordingly, rather than relying on the self-preservation effect of thermodynamically unstable pure CH₄ hydrate, enhancing thermodynamic stability using thermodynamic promoters is expected to offer greater energy efficiency and economic viability and enabling storage under significantly milder temperature and pressure conditions.

Among the promoters reported to date, tetrahydrofuran (THF), cyclopentane (CP), and 1,2-epoxycyclopentane (12ECP) exhibited the most significant thermodynamic promotion performance. Well-known conventional promoters, such as THF and CP, can increase the equilibrium temperature of CH₄ hydrate up to approximately 20 K. In particular, due to its miscibility with water, THF has been reported to function as a kinetic promoter and to enable the formation process without stirring. Meanwhile, 12ECP is not miscible with water but has been reported to exhibit higher promotion performance than THF or CP.

As stated above, efforts to develop new promoters have continued to date. Accordingly, a substantial number of commercially available substances having molecular sizes suitable for sII or sH hydrate structures have already been investigated. Therefore, the discovery of new promoters is expected to approach its limits, and attempts to synthesize novel compounds that are not currently commercialized as promoters yet are considered impractical and economically unviable. Accordingly, for the commercialization of HBES technology, it is necessary to achieve maximum promoter performance by utilizing known cost-effective promoters.

### DISCLOSURE OF THE INVENTION

### Problems to be Solved by the Invention

The present disclosure is to provide a method for achieving synergistic effect for improving thermodynamic stability of gas hydrates by using organic mixture as hybrid promoter. However, problems to be solved by the present disclosure are not limited to the above-described problems. Although not described herein, other problems to be solved by the present disclosure can be clearly understood by a person with ordinary skill in the art from the following description.

### Means for Solving the Problems

A first aspect of the present disclosure provides a hybrid promoter including a first promoter; and a second promoter different from the first promoter, wherein the first promoter is cycloheptanone or cyclooctanone, and the hybrid promoter provides greater thermodynamic stability to a gas hydrate than either the first promoter or the second promoter used alone.

A second aspect of the present disclosure provides a hydrate including the hybrid promoter of the first aspect.

A third aspect of the present disclosure provides an sH-type thermodynamic promoter for clathrate hydrates, wherein the sH-type thermodynamic promoter is cyclooctanone.

### Effects of the Invention

A hybrid promoter according to embodiments of the present disclosure can provide greater performance than conventional stand-alone promoters.

The hybrid promoter according to embodiments of the present disclosure can significantly reduce the energy required for the overall processes of CH₄ hydrate formation, storage, and transportation, thereby enabling an energy-efficient process.

The hybrid promoter according to embodiments of the present disclosure is more cost-effective than a stand-alone cyclooctanone promoter, thereby enhancing the economic viability of the overall process.

### BRIEF DESCRIPTION OF THE DRAWINGS

FIGS. 1A-1E show skeletal structural formulas of compounds (cyclooctenone (FIG. 1A), cycloheptanone (FIG. 1B), cyclooctane (FIG. 1C), methylcyclohexane (FIG. 1D), and 2,2-dimethylbutane (FIG. 1E)) used in an example of the present disclosure.

FIG. 2 is a diagram illustrating an apparatus for preparing CH₄ hydrate by adding a stand-alone promoter and a hybrid promoter, respectively, and for measuring phase equilibrium temperature-pressure conditions of the prepared CH₄ hydrate, according to an embodiment of the present disclosure.

FIG. 3 shows a P-T curve of [cyclooctane (cOct, 1.5 mol %)+cyclooctanone (cOct=O, 1.5 mol %)+CH₄]hydrate according to an example of the present disclosure.

FIG. 4 shows P-T curves of promoters according to an example of the present disclosure and conventional promoters.

FIG. 5A to FIG. 5C show high-resolution powder diffraction (HRPD) patterns measured at 150 K of [cOct (3.0 mol %)+CH₄]hydrate, [cOct=O (3.0 mol %)+CH₄]hydrate, and [cOct (1.5 mol %)+cOct=O (1.5 mol %)+CH₄]hydrate, respectively, according to an example of the present disclosure.

FIG. 6A to FIG. 6C show diagrams representing sizes of cOct and cOct=O molecules, respectively, (FIG. 6A and FIG. 6B) and a schematic diagram of an sH phase formed by (cOct+cOct=O+CH₄) hydrate (FIG. 6C) according to an example of the present disclosure.

FIG. 7 shows solid-state ¹³C NMR spectra measured at 210 K of [cOct (3.0 mol %)+CH₄]hydrate, [cOct=O (3.0 mol %)+CH₄]hydrate, and [cOct (1.5 mol %)+cOct=O (1.5 mol %)+CH₄]hydrate according to an example of the present disclosure.

FIG. 8 shows equilibrium P-T curves of [cOct (3.0 mol %)+CH₄]hydrate, [cOct=O (3.0 mol %)+CH₄]hydrate, and [cOct (1.5 mol %)+cOct=O (1.5 mol %)+CH₄]hydrate according to an example of the present disclosure.

FIG. 9 shows equilibrium P-T curves of [cOct (1.5 mol %)+cOct=O (1.5 mol %)+CH₄]hydrate measured five times over three years according to an example of the present disclosure.

FIG. 10A shows equilibrium P-T curves according to the composition of cOct=O, and FIG. 10B shows T-x₂ curves under isobaric conditions (where x₂ denotes the mol % of cOct=O), respectively, according to an example of the present disclosure.

FIG. 11A and FIG. 11B show PXRD patterns (150 K) and ¹³C NMR spectra (210 K) of (cOct+cOct=O+CH₄) hydrates with various compositions, respectively, according to an example of the present disclosure.

FIG. 12A shows equilibrium P-T curves of CH₄ hydrates including 22DMB, MCH, and [22DMB+MCH], respectively, according to an example of the present disclosure, and FIG. 12B shows equilibrium P-T curves of CH₄ hydrates including MCH, cOct, and [MCH+cOct], respectively, according to an example of the present disclosure.

FIG. 13A shows equilibrium P-T curves of CH₄ hydrates including 22DMB, cOct=O, and [22DMB+cOct=O], respectively, according to an example of the present disclosure, and FIG. 13B shows equilibrium P-T curves of CH₄ hydrates including MCH, cOct=O, and [MCH+cOct=O], respectively, according to an example of the present disclosure.

FIG. 14 shows equilibrium P-T curves of CH₄ hydrates including cyclooctane (cOct), cycloheptanone (cHpt=O), and [cOct+cHpt=O], respectively, according to an example of the present disclosure.

### BEST MODE FOR CARRYING OUT THE INVENTION

Hereafter, embodiments of the present disclosure will be described in detail with reference to the accompanying drawings. However, it is to be noted that the present disclosure is not limited to the embodiments but can be embodied in various other ways. Also, the accompanying drawings are provided to help easily understand the embodiments of the present disclosure and the technical conception described in the present disclosure is not limited by the accompanying drawings. In the drawings, parts irrelevant to the description are omitted for the simplicity of explanation, and the size, form and shape of each component illustrated in the drawings can be modified in various ways. Like reference numerals denote like parts through the whole document.

Through the whole document, the term “connected to” or “coupled to” that is used to designate a connection or coupling of one element to another element includes both a case that an element is “directly connected or coupled to” another element and a case that an element is “electronically connected or coupled to” another element via still another element.

Through the whole document, the term “on” that is used to designate a position of one element with respect to another element includes both a case that the one element is adjacent to the other element and a case that any other element exists between these two elements.

Further, through the whole document, the term “comprises or includes” and/or “comprising or including” used in the document means that one or more other components, steps, operation and/or existence or addition of elements are not excluded in addition to the described components, steps, operation and/or elements unless context dictates otherwise.

Through the whole document, the term “about or approximately” or “substantially” is intended to have meanings close to numerical values or ranges specified with an allowable error and intended to prevent accurate or absolute numerical values disclosed for understanding of the present disclosure from being illegally or unfairly used by any unconscionable third party.

Through the whole document, the term “step of” does not mean “step for”.

Through the whole document, the term “combination of” included in Markush type description means mixture or combination of one or more components, steps, operations and/or elements selected from a group consisting of components, steps, operation and/or elements described in Markush type and thereby means that the disclosure includes one or more components, steps, operations and/or elements selected from the Markush group.

Through this whole specification, a phrase in the form “A and/or B” means “A or B, or A and B”.

Hereinafter, embodiments and examples of the present disclosure will be described in detail with reference to the accompanying drawings. However, the present disclosure may not be limited to the following embodiments, examples, and drawings.

A first aspect of the present disclosure provides a hybrid promoter including a first promoter; and a second promoter different from the first promoter, wherein the first promoter is cycloheptanone or cyclooctanone, and the hybrid promoter may provide greater thermodynamic stability to a gas hydrate than either the first promoter or the second promoter used alone.

In an embodiment of the present disclosure, the first promoter and the second promoter may be sI-type, sII-type, or sH-type thermodynamic promoters, and the first promoter and the second promoter may be the same type of thermodynamic promoter.

In an embodiment of the present disclosure, the second promoter may be an organic compound.

In an embodiment of the present disclosure, the second promoter may be a conventional promoter.

In an embodiment of the present disclosure, the second promoter may be selected from the group consisting of C₃₋₁₀ alkanes substituted or unsubstituted with one or more C₁₋₄ alkyl groups; C₅₋₁₀ cycloalkanones; C₅₋₁₀ cycloalkanes substituted or unsubstituted with one or more C₁₋₄ alkyl groups; tetrahydrofuran; tetrahydrothiophene; and tert-butyl methyl ether.

In an embodiment of the present disclosure, the second promoter may be selected from the group consisting of propane, butane, 2,2-dimethylbutane, pentane, hexane, heptane, octane, nonane, decane, cyclopentanone, cyclohexanone, cycloheptanone, cyclooctanone, cyclononanone, cyclodecanone, cyclopentane, cyclohexane, cycloheptane, cyclooctane, cyclononane, cyclodecane, methylcyclohexane, tetrahydrofuran, tetrahydrothiophene, 2-methylbutane, 2,3-dimethylbutane, 2,2,3-dimethylbutane, 2,2-dimethylpentane, 2,3-dimethylpentane, 3,3-dimethylpentane, methylcyclopentane, ethylcyclopentane, 1,1-dimethylcyclohexane, 1,2-dimethylcyclohexane, 1,3-dimethylcyclohexane, 1,4-dimethylcyclohexane, ethylcyclohexane, and tert-butyl methyl ether.

In an embodiment of the present disclosure, a molar ratio of the first promoter to the second promoter (first promoter:second promoter) may be about 1:10 to about 5:1. In an embodiment of the present disclosure, a molar ratio of the first promoter to the second promoter may be about 1:10 to about 5:1, about 1:9 to about 5:1, about 1:8 to about 5:1, about 1:7 to about 5:1, about 1:6 to about 5:1 about 1:5 to about 5:1, about 1:5 to about 4:1, about 1:5 to about 3:1, about 1:5 to about 2:1, about 1:2 to about 5:1, about 1:2 to about 2:1. In an embodiment of the present disclosure, a molar ratio of the first promoter to the second promoter may be about 1:1.

A second aspect of the present disclosure provides a hydrate including the hybrid promoter of the first aspect.

Detailed descriptions of the second aspect of the present disclosure, which overlap with those of the first aspect of the present disclosure, are omitted hereinafter, but the descriptions of the first aspect of the present disclosure may be identically applied to the second aspect of the present disclosure, even though they are omitted hereinafter.

In an embodiment of the present disclosure, the hydrate may have an sH structure.

In an embodiment of the present disclosure, the hybrid promoter may be contained in an amount of about 1 mol % to about 6 mol %, but is not limited thereto. In an embodiment of the present disclosure, the hybrid promoter may be contained in an amount of about 1 mol % to about 6 mol %, about 1 mol % to about 5 mol %, about 1 mol % to about 4 mol %, about 1 mol % to about 3 mol %, about 1 mol % to about 2 mol %, about 2 mol % to about 6 mol %, about 2 mol % to about 5 mol %, about 2 mol % to about 4 mol %, about 2 mol % to about 3 mol %, about 3 mol % to about 6 mol %, about 3 mol % to about 5 mol %, about 3 mol % to about 4 mol %, about 4 mol % to about 6 mol %, about 4 mol % to about 5 mol %, or about 5 mol % to about 6 mol %, but may not be limited thereto. For example, when the first promoter and the second promoter are sH-type thermodynamic promoters, the hybrid promoter may be contained in an amount of about 3 mol %, and when the first promoter and the second promoter are sII-type thermodynamic promoters, the hybrid promoter may be contained in an amount of about 5 mol % to about 6 mol %, or about 5.6 mol %.

A third aspect of present disclosure provides an sH-type thermodynamic promoter for clathrate hydrates, wherein the sH-type thermodynamic promoter is cyclooctanone.

Detailed descriptions of the third aspect of the present disclosure, which overlap with those of the first and the second aspect of the present disclosure, are omitted hereinafter, but the descriptions of the first and the second aspect of the present disclosure may be identically applied to the third aspect of the present disclosure, even though they are omitted hereinafter.

Hereinafter, the present disclosure will be explained in more detail with reference to Examples. However, the following Examples are illustrated only for better understanding of the present disclosure but do not limit the present disclosure.

### MODE FOR CARRYING OUT THE INVENTION

### Examples

### <Used Materials>

TABLE 1

| | | | | | Product | |
|---|---|---|---|---|---|---|
| IUPAC name | abbreviation | CAS No. | MW | Supplier | number | Purity |
| Cyclooctanone | cOct═O | 502-49-8 | 126.20 | TCI(Tokyo | C0504 | 97+% |
| | | | | Chemical | | |
| | | | | Industry) | | |
| Cyclooctane | cOct | 292-64-8 | 112.17 | Alfa Aesar | L15000 | 99+% |
| Methylcyclohexane | MCH | 108-87-2 | 98.19 | TCI | M0190 | 99+% |
| 2,2-dimethylbutane | 22DMB | 75-83-2 | 86.18 | TCI | D0689 | 98+% |
| cycloheptanone | cHpt═O | 502-42-1 | 112.17 | TCI | C0466 | 98+% |
| Water | H₂O | 7732-18-5 | 18.02 | Merck | 115333 | LC-MS |
| Methane | CH₄ | 74-82-8 | 16.04 | Daesung Gas | | 99.95% |

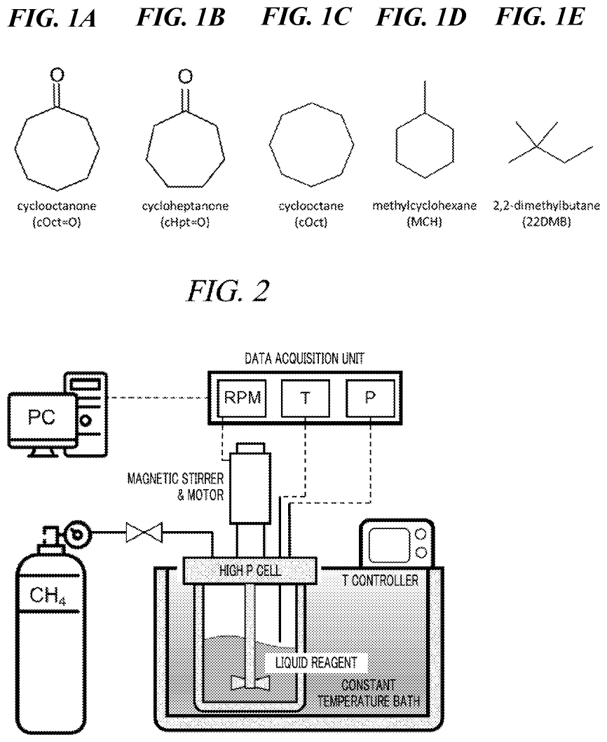

The chemicals used in the present example are shown in FIGS. 1A-1E and Table 1. All the chemicals were used as received without further purification.

### <P-T Trace Curve Derivation>

A P-T trace curve was obtained through the following procedure, and the equilibrium P-T conditions were determined from this curve:

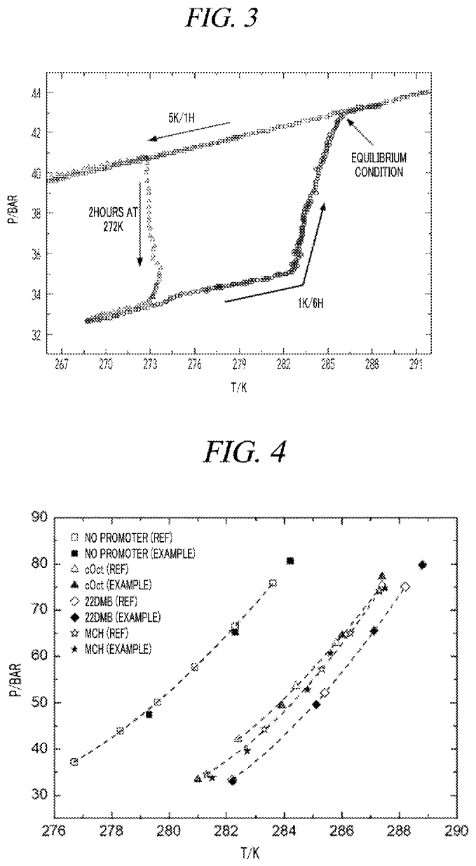

(1) 1.12 g of cOct, 1.26 g of cOct=O, and 1.19 g of [cOct (1.5 mol %)+cOct=O (1.5 mol %)] were added to 6.12 g of H₂O to achieve a total sH former concentration of 2.9 mol % to 3.0 mol %. The mixture was placed in a high-pressure resistant cell (~100 mL), and CH₄ gas was injected at ambient temperature (~290 K) to a desired pressure; (2) the temperature of the cell was increased to 313 K with stirring at 150 rpm to ensure cOct and cOct=O were in a fully liquid state; (3) the cell was cooled to 263 K for 10 hours (−5 K/h), then heated to 272 K and maintained for 2 hours (although a pressure drop occurs near 273 K even at slower cooling rates below −1 K/h, sufficiently supercooling followed by maintaining a temperature just below the freezing point (273 K) is much less time-consuming); (4) when no further pressure decrease was observed, the temperature of the cell was lowered to 268 K to confirm completion of hydrate formation; (5) equilibrium P-T conditions were obtained by gradually increasing the temperature at a very slow rate of +0.15 K/h (+1 K/6 h). Pressure (P) and temperature (T) inside the cell were measured using a pressure transmitter (model A-10, WIKA, Germany) and a resistance temperature detector (Pt100-class B), respectively. The P and T data were automatically recorded at 1-minute intervals until the temperature reached 293 K. During the processes (2) to (5), the stirring rate was maintained at 150 rpm. The resulting P-T curve (FIG. 3) was consistent with the conventional P-T curve (FIG. 4), confirming the integrity of the measurement method and data in the present example.

### <Hydrate Analysis>

After the processes (1) to (4), solid samples were rapidly collected from the cell immersed in liquid nitrogen. The samples were finely ground (d<200 μm) and stored in liquid nitrogen until measurement. The crystal structures of the powdered samples were analyzed using synchrotron high-resolution powder diffraction (HRPD) at the Pohang Accelerator Laboratory (beamline 9B) or powder X-ray diffraction (PXRD) at the KAIST Analysis Center (Smart Lab, Rigaku). HRPD patterns were obtained using X-rays with a wavelength of

$$λ = 1.5425 Å$$

over a 26 range of 5.0° to 125.0°. The measurement was performed with a step interval of 0.01° and an exposure time of 0.7 s. Solid-state ¹³C NMR experiments were performed to analyze the distribution of guest molecules using a Bruker Avance II 400 MHz NMR spectrometer at the Korea Basic Science Institute (Seoul Western Center). ¹³C high-power decoupling/magic-angle spinning (MAS) NMR spectra were obtained using a 4 mm OD zirconia rotor with an MAS rate of 5 kHz. A pulse length of 1.6 ρs and a repetition delay of 3 s were applied. The static ¹³C signal of tetramethylsilane at room temperature was used as a reference (0 ppm).

### 1. High Resolution Powder Diffractometer (HRPD)

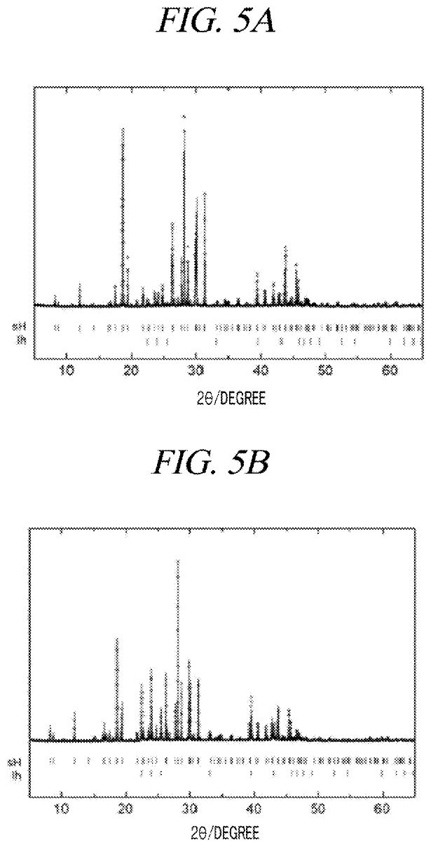

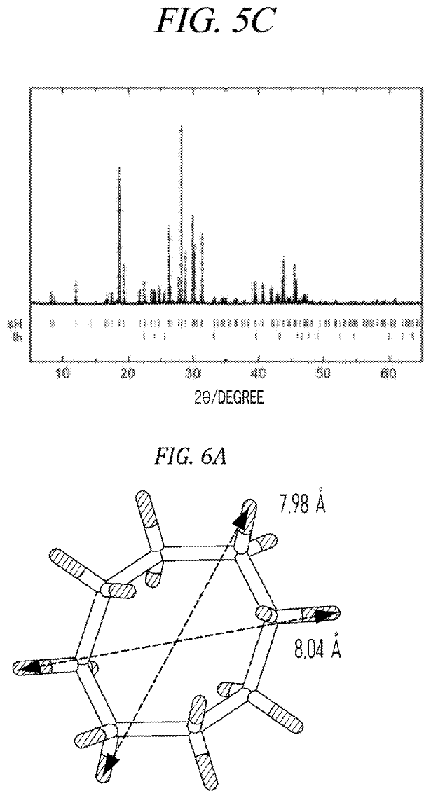

Referring to FIG. 5A, in the case of (cOct+CH₄) hydrate, an sH structure (P6/mmm) with lattice parameters of

$$a = 12.275 Å and c = 9.982 Å$$

at 150 K was observed. These experimental values are in close agreement with the parameters

$$( a = 12.3 \; 13 Å and c = 10.0 \; 54 Å at 215 K )$$

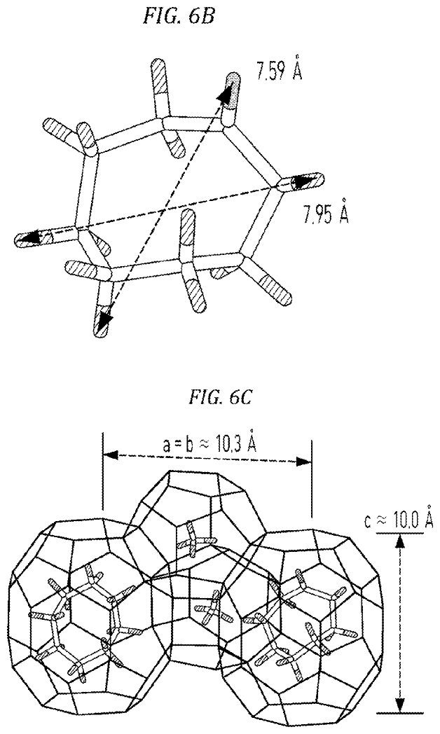

reported in the prior literature: K. A. Udachin et al., “Transformation of the hexagonal-structure clathrate hydrate of cyclooctane to a low-symmetry from below 167 K”, Angew. Chem. Int. Ed. 47 (2008) 9704-9707. Referring to FIG. 6A to FIG. 6C, cOct and cOct=O have similar molecular sizes, and, thus, cOct=O is also anticipated to facilitate the formation of an sH hydrate.

Referring to FIG. 5B, in the case of (cOct=O+CH₄) hydrate, an sH structure with lattice parameters of

$$a = 12.2 \; 95 Å and c = 10.0 \; 00 Å$$

was observed.

Referring to FIG. 5C, in the case of [cOct (1.5 mol %)+cOct=O (1.5 mol %)+CH₄]hydrate, the lattice parameters of

$$a = 12.2 \; 79 Å and c = 9.9 \; 84 Å$$

fall between those of (cOct+CH₄) and (cOct=O+CH₄) hydrates. In particular, rather than forming discrete phases of (cOct+CH₄) hydrate (with relatively smaller lattice parameters) and (cOct=O+CH₄) hydrate (with relatively larger lattice parameters), a single homogenous phase with one set of lattice parameters was observed.

### 2. ¹³C NMR

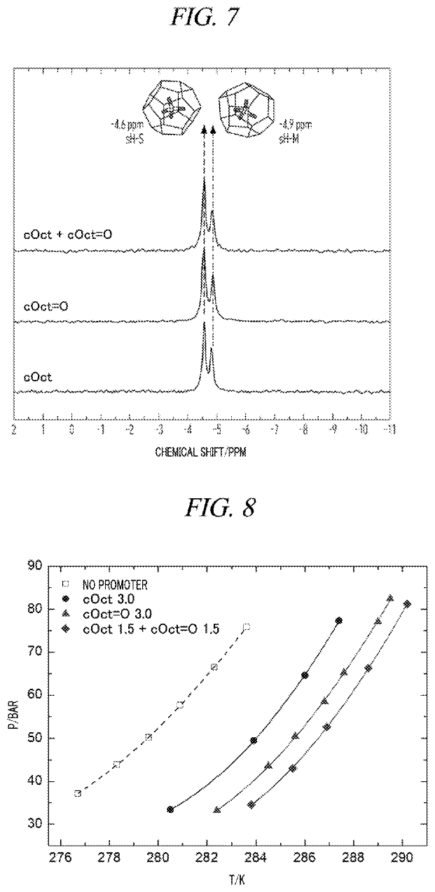

Solid-state ¹³C NMR analysis (see FIG. 7) reveals two distinct peaks at approximately −4.6 ppm and −4.9 ppm, corresponding to CH₄ molecules trapped within the sH-S and sH-M cages, respectively. The area ratios (A_{S}/A_{M}) of the two peaks for (cOct+CH₄), (cOct=O+CH₄), and (cOct+cOct=O+CH₄) hydrates are 1.49, 1.47, and 1.58, respectively, which are close to the theoretical value of 1.5 for sH hydrates. Since no peak corresponding to CH₄ in sH-L cages was observed, it can be inferred that sH-L cages are exclusively occupied by cOct or cOct=O. FIG. 6C illustrates a uniform and homogeneous sH phase formed by (cOct+cOct=O+CH₄) hydrate.

### 3. Equilibrium P-T Curve

FIG. 8 illustrates the four-phase (L₁-L₂-H-V) equilibrium P-T conditions for the (cOct/cOct=O+CH₄) hydrate system. At a constant pressure of 50 bar, the addition of cOct increases the equilibrium temperature of CH₄ hydrate by about 4.5 K (circle markers), while the addition of cOct=O increases the equilibrium temperature by about 6.0 K (triangle markers). This indicates that the novel sH former cOct=O exhibits superior promotion performance compared to the conventional promoter cOct. In contrast, the addition of a 1:1 mixture of cOct and cOct=O increases the equilibrium temperature by 7.0 K (diamond markers). In general, a mixture of two different promoters is anticipated to exhibit an intermediate promotion performance between the two promoters. However, the mixture of cOct and cOct=O shows superior promotion performance even compared to stand-alone cOct=O, which is the more effective of the two individual promoters. This indicates that cOct and cOct=O synergistically enhance the thermodynamic stability of CH₄ hydrate.

In view of the above phenomenon, the following two critical aspects should be considered:

### (1) Consideration of Measurement Error or Reliability of Equilibrium Condition

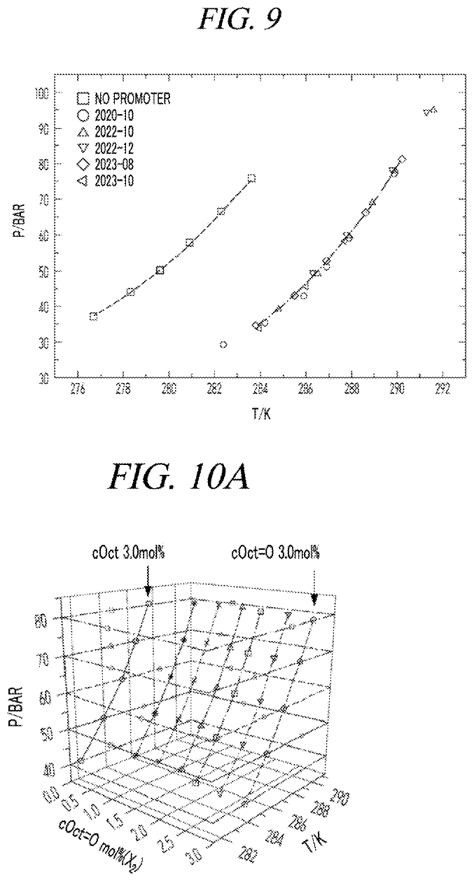

As described above, the equilibrium conditions of pure CH₄ and (cOct+CH₄) hydrate are consistent with previously reported data. Referring to FIG. 9, in the case of (cOct+cOct=O+CH₄) hydrate, five equilibrium curves were obtained over three years, all of which coincide, confirming that the observed synergistic promotion is not attributable to measurement error.

(2) Consideration of Dissociation Behavior of (cOct+cOct=O+CH₄) Hydrate

As shown in FIG. 7, although cOct and cOct=O exhibit different promotion performances, no distinct shoulder is observed during the dissociation process of (cOct+cOct=O+CH₄) hydrate (between 286 K and 290 K, as indicated by the circle markers (1K/6H) in FIG. 3). That is, instead of the sequential dissociation of (cOct=O+CH₄) hydrate followed by (cOct+CH₄) hydrate, the (cOct+cOct=O+CH₄) hydrate dissociates as a single uniform phase. Combined with the HRPD results, this indicates that cOct and cOct=O behave as if they were a single type of promoter in forming sH CH₄ hydrate.

TABLE 2

| x₂ = 0 | | x₂ = 0.5 | | x₂ = 1.0 | | x₂ = 1.5 | | x₂ = 2.0 | | x₂ = 2.5 | | x₂ = 3 | |
|---|---|---|---|---|---|---|---|---|---|---|---|---|---|
| T/K | P/bar | T/K | P/bar | T/K | P/bar | T/K | P/bar | T/K | P/bar | T/K | P/bar | T/K | P/bar |
| 280.5 | 33.4 | 282.9 | 32.9 | 283.7 | 33.5 | 283.8 | 34.6 | 282.4 | 32.4 | 282.4 | 32.9 | 282.9 | 33.2 |
| 283.9 | 49.5 | 285.8 | 45.3 | 286.1 | 45.5 | 285.5 | 43.0 | 285.1 | 45.0 | 284.8 | 44.0 | 285.0 | 43.6 |
| 286.0 | 64.6 | 287.5 | 58.0 | 287.6 | 58.2 | 286.9 | 52.7 | 286.9 | 58.0 | 286.6 | 55.1 | 286.1 | 50.5 |
| 287.4 | 77.3 | 289.0 | 71.0 | 288.9 | 69.4 | 288.6 | 66.3 | 288.5 | 71.5 | 288.1 | 68.3 | 287.3 | 58.5 |
| | | 290.3 | 84.6 | 290.5 | 83.7 | 290.2 | 81.2 | 289.8 | 83.5 | 289.9 | 83.3 | 288.1 | 65.2 |
| | | | | | | | | | | | | 289.5 | 77.1 |
| | | | | | | | | | | | | 290 | 82.5 |

* x₂ denotes mol % of cOct=O. The total fraction of cOct and cOct=O is 3.0 mol %.

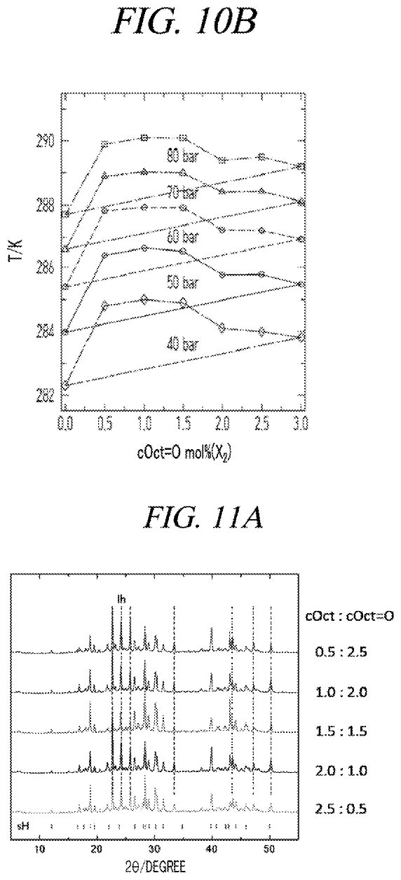

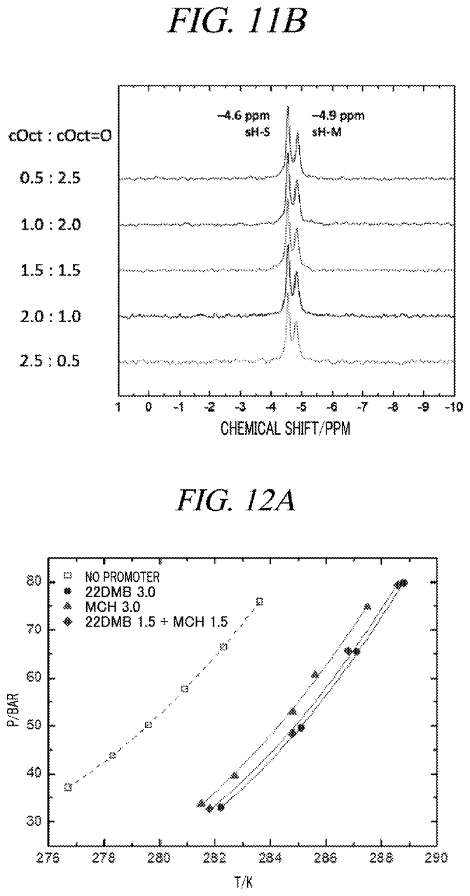

The table 2 shows equilibrium P-T conditions measured for various compositions of cOct=O (x₂), while maintaining the total fraction of cOct and cOct=O at 3.0 mol %. Referring to FIG. 10A, the data provided in Table 2 are plotted as x₂-T-P diagrams within the pressure range of 40 bar to 80 bar. Referring to FIG. 10B, the figure illustrates the isobaric equilibrium curves as a function of the mixing ratio of cOct and cOct=O. A straight line connecting T values at x₂=0 (pure cOct) and x₂=3.0 (pure cOct=O) represents the expected dissociation temperature assuming an ideal mixing effect. At all pressures, cOct and cOct=O exhibit synergistic promotion, with maximum promotion performance in the range of x₂=0.5 mol % to 1.5 mol %. Within this composition range, a synergistic promotion performance was observed, with equilibrium T values approximately 2 K greater than the values calculated assuming an ideal mixing effect. Also, the equilibrium T values are approximately 1 K higher than that of (cOct=O+CH₄) hydrate (x₂=3.0 mol %). Referring to FIG. 11A and FIG. 11B, the PXRD patterns and ¹³C NMR spectra of the hydrates were analyzed at various compositional ratios of cOct and cOct=O, confirming that all hydrates in FIG. 10 have the sH structure.

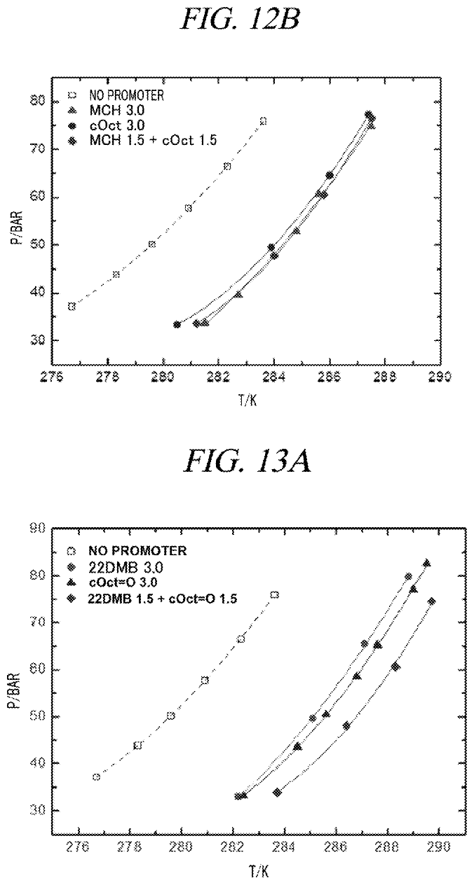

To determine whether the observed synergistic promotion is specific to the (cOct+cOct=O) system or represents a general phenomenon, the equilibrium curves of CH₄ hydrates comprising three representative promoters (22DMB, MCH, and cOct) and their mixtures were analyzed (FIG. 12A and FIG. 12B). As shown in FIG. 12A, a 1:1 mixture of 22DMB and MCH exhibited promotion performance between those of the individual promoters. Similarly, as shown in FIG. 12B, a 1:1 mixture of MCH and cOct also exhibited promotion performance comparable to the individual promoters. None of these mixtures were superior to their constituent individual promoters.

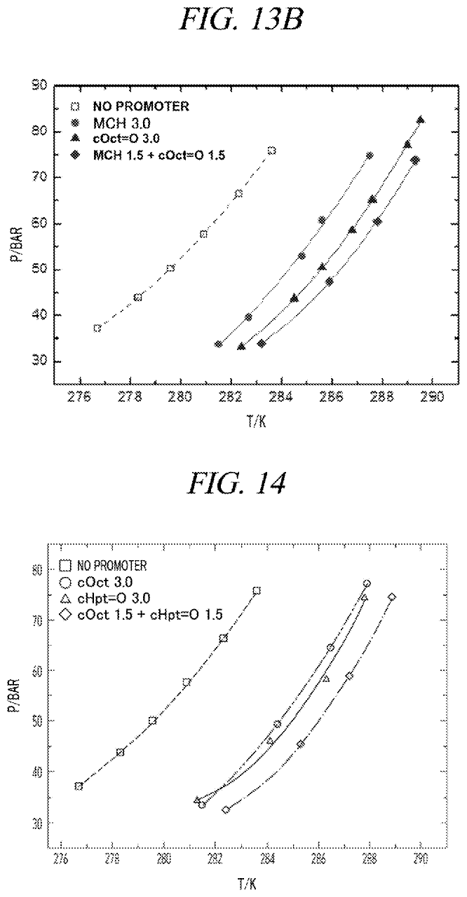

However, this phenomenon is not limited to the (cOct+cOct=O) system. Referring to FIG. 13A, synergistic promotion was also observed when 22DMB was mixed with cOct=O. Although 22DMB exhibits lower promotion performance than cOct=O, a 1:1 mixture of 22DMB and cOct=O enhanced the equilibrium temperature by 1.1 K (at 50 bar) compared to pure cOct=O. Similarly, Referring to FIG. 13B although MCH exhibits lower performance than cOct=O, a 1:1 mixture of MCH and cOct=O enhanced the equilibrium temperature by 0.7 K (at 50 bar) compared to pure cOct=O. Further, although cOct and cHpt=O exhibit comparable promotion performances when used individually, a 1:1 mixture (1.5 mol %:1.5 mol %) enhances the equilibrium temperature by 1.0 K to 1.5 K compared to the individual promoters, indicating synergistic promotion (see FIG. 14).

### <Economic Analysis>

From an engineering perspective, these hybrid promoters are anticipated to be applicable to various fields utilizing hydrates. The promotion performance of cOct=O is higher than that of cOct. However, mixtures of cOct and cOct=O at ratios of 1:1, 2:1, or 5:1 (i.e., x₂=1.5 mol %, 1.0 mol %, or 0.5 mol %) exhibit significantly higher promotion performances than stand-alone cOct=O. A 1 K increase in the equilibrium temperature over a pressure range of 40 bar to 80 bar and a temperature range of 280 K to 290 K is equivalent to an equilibrium pressure reduction of 7 bar to 10 bar. This ultimately means that the pressure required for storing gas in hydrate form and for storing and transporting the formed hydrate can be reduced by about 10 bar.

Furthermore, as shown in Table 3 below, since cOct is significantly more cost-effective than cOct=O, the hybrid promoter offers a distinct economic advantage over the use of cOct=O alone. For example, assuming that the price of cOct is 30% of that of cOct=O (e.g., cOct (Merck) at $300/kg and cOct=O (ThermoFisher) at $964/kg), a 2:1 hybrid promoter is 50% less expensive than a single cOct=O promoter. Accordingly, the synergistic promotion performance of the hybrid promoter maximizes both energy efficiency and economic feasibility, thereby enabling sustainable hydrate-based gas storage (HBGS).

TABLE 3

| | Supplier | | |
|---|---|---|---|
| | TCI | ThermoFisher | Merck |
| cOct | $27.0/25 mL | $113/250 mL | $630/2.5 L |
| cOct = O | $39.0/25 g | $482/500 g | $131/100 g |
| Ratio (cOct/cOct = O) | 82% | 56% | 23% |

** The price per kilogram ratio above was calculated based on a density of 0.84 kg/L for cOct.

It would be understood by a person with ordinary skill in the art that various changes and modifications may be made based on the above description without changing technical conception and essential features of the present disclosure. Thus, it is clear that the embodiments are illustrative in all aspects and do not limit the present disclosure. The scope of the present disclosure is defined by the following claims. It shall be understood that all modifications and embodiments conceived from the meaning and scope of the claims and their equivalents are included in the scope of the present disclosure.

The scope of the present disclosure is defined by the following claims rather than by the detailed description of the embodiment. It shall be understood that all modifications and embodiments conceived from the meaning and scope of the claims and their equivalents are included in the scope of the present disclosure.

What is claimed is:

1. A hybrid promoter, comprising:
a first promoter; and
a second promoter different from the first promoter,
wherein the first promoter is cycloheptanone or cyclooctanone, and
the hybrid promoter provides greater thermodynamic stability to a gas hydrate than either the first promoter or the second promoter used alone.

2. The hybrid promoter of claim 1,
wherein the first promoter and the second promoter are sI-type, sII-type, or sH-type thermodynamic promoters, and
the first promoter and the second promoter are the same type of thermodynamic promoter.

3. The hybrid promoter of claim 1,
wherein the second promoter is an organic compound.

4. The hybrid promoter of claim 3,
wherein the second promoter is selected from the group consisting of C₃₋₁₀ alkanes substituted or unsubstituted with one or more C₁₋₄ alkyl groups; C₅₋₁₀ cycloalkanones; C₅₋₁₀ cycloalkanes substituted or unsubstituted with one or more C₁₋₄ alkyl groups; tetrahydrofuran; tetrahydrothiophene; and tert-butyl methyl ether.

5. The hybrid promoter of claim 3,
wherein the second promoter is selected from the group consisting of propane, butane, 2,2-dimethylbutane, pentane, hexane, heptane, octane, nonane, decane, cyclopentanone, cyclohexanone, cycloheptanone, cyclooctanone, cyclononanone, cyclodecanone, cyclopentane, cyclohexane, cycloheptane, cyclooctane, cyclononane, cyclodecane, methylcyclohexane, tetrahydrofuran, tetrahydrothiophene, 2-methylbutane, 2,3-dimethylbutane, 2,2,3-dimethylbutane, 2,2-dimethylpentane, 2,3-dimethylpentane, 3,3-dimethylpentane, methylcyclopentane, ethylcyclopentane, 1,1-dimethylcyclohexane, 1,2-dimethylcyclohexane, 1,3-dimethylcyclohexane, 1,4-dimethylcyclohexane, ethylcyclohexane, and tert-butyl methyl ether.

6. The hybrid promoter of claim 1,
wherein a molar ratio of the first promoter to the second promoter (first promoter:second promoter) is 1:10 to 5:1.

7. A hydrate, comprising:
the hybrid promoter of claim 1.

8. The hydrate of claim 7,
wherein the hydrate has an sH structure.

9. The hydrate of claim 7,
wherein the hybrid promoter is contained in an amount of 1 mol % to 6 mol %.

10. An sH-type thermodynamic promoter for clathrate hydrates,
wherein the sH-type thermodynamic promoter is cyclooctanone.
